(19) United States
(12) Patent Application Publication
(10) Pub. No.: US 2012/0149784 A1
(43) Pub. Date: Jun. 14, 2012

(54) TABLETTING OF ERYTHRITOL AND ISOMALT

(75) Inventors: Catherine Patricia L. Boghmans, Beigem (BE); Liesbeth Maria Fernande Meeus, Everberg (BE)

(73) Assignee: CARGILL ,INCORPORATED, Wayzata, MN (US)

(21) Appl. No.: 13/391,007

(22) PCT Filed: Jul. 12, 2010
(86) PCT No.: PCT/EP2010/004223

§ 371 Date: Feb. 17, 2012

(30) Foreign Application Priority Data
Aug. 18, 2009 (EP) 09010597.4

(51) Int. Cl. A23L 1/09; A23P 1/02; B02C 23/18; A61K 47/26; A61K 8/60

(52) U.S. Cl. 514/777; 241/15; 426/650; 426/285

(57) ABSTRACT

Erythritol is granulated together with from 10% w/w to 50% w/w isomalt. Chewable tablets are prepared and the corresponding process is described.

### TECHNICAL FIELD

The present invention relates to the preparation of an erythritol and isomalt containing composition suitable for tabletting.

### BACKGROUND OF THE INVENTION

With the present interest in the use of sugar-free and/or low calorie products, tablets for pharmaceutical, confectionery or food applications are mostly made with sugar alcohols, such as xylitol, maltitol, sorbitol, mannitol and erythritol.

The tablet does not only contain the drug or a reagent, it also contains other ingredients which act as fillers, such as lactose or phosphates; lubricating agents, such as talc, stearic acid or paraffin and disintegrating agents, such as carboxymethyl-cellulose or starch. For confectionery purposes the tablets often include aroma's and colorants at low concentration.

Direct compression of spray-dried erythritol has been described in European patent EP 0 497 439. The tablets are always prepared with maltodextrin as binder.

European patent application EP 0 528 604 discloses the co-crystallized sorbitol and xylitol and tablets made therefrom.

EP 0 896 528 relates to a polyol composition with high concentration of a non-hygroscopic polyol obtained by spray-drying or fluidized bed granulation.

EP 0 922 464 relates to a process for preparing quickly disintegradable compression-molded materials based upon erythritol. A tablet is obtained by direct compression molding. The thus obtained quickly disintegradable compression molded material is endowed with excellent disintegration and dissolution properties when put in the oral cavity or water.

EP 0 913 148 relates to a process for preparing an erythritol containing composition suitable for use as an excipient for tabletting. The suitable composition was prepared by co-crystallization of erythritol and a second polyol such as sorbitol. The erythritol was used as such and mixed with sorbitol before co-crystallisation. After the co-crystallisation, the product was milled and tabletted. The process does not involve a granulation step.

There is a further interest for using erythritol and isomalt as excipients in tablets.

### SUMMARY OF THE INVENTION

The current invention relates to granulated compressible composition consisting of erythritol and less than 50% w/w isomalt and at least 10% w/w isomalt, preferably at least 15% w/w isomalt, more preferably at least 20% w/w.

It further relates to a chewable tablet comprising the previously described compressible composition.

Furthermore it relates to a process for preparing the compressible composition of the current invention and it is comprising the following steps:

a) taking erythritol,

b) adding isomalt in dry or liquid form, optionally adding water

c) granulating,

d) optionally wet sieving of granulated product,

e) drying the granulated product,

f) optionally sieving of the granulated product.

It further describes a process for preparing the tablet according to the current invention and it comprises the following steps:

a) Taking the granulated product prepared according to the current invention

b) Blending with a lubricant,

c) Tabletting at compressing forces from 5 to 20 kN.

Finally it relates to the use of tablet in food, feed, pharma and cosmetic applications.

### DETAILED DESCRIPTION OF THE INVENTION

The current invention relates to granulated compressible composition consisting of erythritol and less than 50% w/w isomalt and at least 10% w/w isomalt, preferably at least 15% w/w isomalt, more preferably at least 20% w/w.

Erythritol is a tetriitol which is obtainable via chemical processes, preferably other than hydrogenation of carbohydrates, and/or microbial processes or fermentation, preferably fermentation. Any grade of erythritol is suitable and without any limitation, a suitable source of erythritol is a micronized erythritol prepared as described in WO2009016133, or a fine grade of erythritol, or preferably turbomilled erythritol and the like. Mixtures of different grades can be applied as well.

Isomalt is understood to refer to an almost equimolar mixture of 6-glucopyranosyl-sorbitol (6-GPS) and 1-glucopyranosyl-mannitol (1-GPM), and the weight percentage can vary between 43 to 57% of 6-GPS to 57% to 43% of 1-GPM. Any other ratio of both components is falling under the definition of the mixture containing 6-glucopyranosyl-sorbitol, and 1-glucopyranosyl-mannitol. These mixtures can be enriched in one of the component, be it 1-GM or 6-GPS or another isomer, 1-glycopyranosyl-sorbitol (1-GPS) may be present as well. The mixtures containing 6-glucopyranosyl-sorbitol, and/or 1-glucopyranosyl-mannitol, as well as the isomalt may further comprise minor amounts of other substances such mannitol, sorbitol, hydrogenated or non-hydrogenated oligosaccharides as well as optionally glucose, fructose and/or sucrose, trehalulose, isomaltulose or isomaltose. Preferably isomalt containing an almost equimolar mixture of 6-glucopyranosyl-sorbitol (6-GPS) and 1-glucopyranosyl-mannitol (1-GPM) is used. Isomalt is present in an amount of at least 10% w/w, preferably at least 15% w/w, more preferably at least 20% w/w and preferably in an amount less than 50% w/w.

Granulation methods can be divided in two basic types, namely wet methods, which use a liquid in the process, and dry methods in which no liquid is used. Wet granulation is most often used and involves many steps, including: agglomerating (granulating) of dry primary powder particles of active ingredients and excipients in the presence of a granulating fluid upon agitation using low-shear or high-shear mixers or fluidized beds, wet sieving (wet screening) to remove larger lumps, drying the granulated product, and milling or sieving (screening) the dried granulated product to achieve a granulated product having the desired granule size distribution. The obtained granulated product may subsequently be tabletted.

Preferably erythritol is having a specific surface area greater than 0.25 m²/g, preferably greater than 0.3 m²/g and more preferably greater than 0.4 m²/g.

The specific surface area is measured with BET method.

Surprisingly it was found that the specific surface area has an additional positive effect on the subsequent granulation, even with a binder in liquid form. The bigger the specific surface area the better the granulation is performed. Granulation is a process in which primary powder particles are made to form larger entities called granules. The granulation allows preventing segregation of the constituents of the powder mix, to improve the flow properties of the powder mix, and to improve the compaction characteristics of the powder mix.

Furthermore the erythritol is having a volume mean diameter, reference to Ph.Eur.VI, of less than 100 μm, preferably less than 50 μm, more preferably less than 40 μm.

Isomalt is acting as a binder and can be added in dry or liquid form. The preferred binder is isomalt containing an almost equimolar mixture of 6-glucopyranosyl-sorbitol (6-GPS) and 1-glucopyranosyl-mannitol (1-GPM). Liquid isomalt is further containing 1-glycopyranosyl-sorbitol (1-GPS) in quantities of at least 2% based on dry matter.

The composition is further characterized in that it has a moisture pick-up below 1%, preferably below 0.5% at 65% relative humidity, at 25° C.

Furthermore, the current invention relates to the use in food applications, feed, pharma applications, cosmetics, detergents, fertilizer or agrochemical products. In fact, without being limiting, the compressible composition of the current invention can be used in food products, animal feed, health food, dietetic products, animal medicine, with bath agent, in agrochemical products, with fertilizer, with plant granules, with plant seeds or seed grains, and any other product being it ingested by humans and/or animals or any other product which can benefit from the improved properties of the compressible composition of the current invention. The compressible composition of the current invention can be used as carrier for additives based on enzymes or microorganisms, detergent tablets, vitamins, flavors, perfumes, acids, sweeteners or various active ingredients with medicinal or non-medicinal applications. Eventually mixtures of additives can be applied.

It further relates to a chewable tablet comprising the previously described compressible composition. The term “tablet”, as used herein, includes any tablet, in particular tablets in any form, shape and of any physical, chemical or sensory property, and tablets for any route of administration, indication and application. The tablets produced according to the invention is a chewable tablet. A chewable tablet according to the present invention is a tablet where chewing helps to break the tablet particles and release the active ingredient, flavor, aroma or the like, in the mouth before swallowing. Chewable tablets are designed to be mechanically disintegrated in the mouth.

A chewable tablet dosage form can be a pill, tablet, gum and more recently “chewy squares”. The tablet hardness and friability are highly important properties of a chewable tablet comprising active ingredient(s) and having desirable chewability properties.

Said tablets can be applied in food, feed, cosmetics, detergents and/or pharma applications. The chewable tablet is significant different from a quickly disintegradable tablet in the oral cavity or in water and has a different purpose to serve.

As a lubricant agent in tablet formation, magnesium stearate, calcium stearate, stearic acid, sucrose fatty acid esters, and/or talc and the like can be added according to needs. Furthermore surface active agents such as sodium lauryl sulfate, propylene glycol, sodium dodecanesulfonate, sodium oleate sulfonate, and sodium laurate mixed with stearates and talc, sodium stearyl fumarate, sucrose fatty acid esters, and the like can be added according to needs.

The thus obtained tablets have a friability of 0.1 to 0.4%, at a compression force from 5 to 20 kN, preferably from 0.15 to 0.3% according to Ph. Eur. VI. Preferably these tablets have a surface of at least 1 cm² and a weight of 350 mg.

The tensile strength of these tablets can be expressed in function of compression force. A tensile strength at 20 kN of at least 3.5 N/mm², preferably, at least 3.6 N/mm², more preferably at least 3.7 N/mm², most preferably at least 3.8 N/mm² is obtainable.

The tablets have a hardness of at least 140 N, preferably at least 150 N, more preferably at least 155 N at a compression force of 15 kN. Preferably the tablets have a surface of at least 1 cm² and a weight of 350 mg.

The chewable tablets of the current invention have a friability of 0.1 to 0.4%, at a compression force from 5 to 20 kN, and a tensile strength of up to at least 3.8 N/mm².

Furthermore it relates to a process for preparing the compressible composition of the current invention and it is comprising the following steps:

a) taking erythritol,

b) adding isomalt in dry or liquid form, optionally adding water

c) granulating,

d) optionally wet sieving of granulated product,

e) drying the granulated product,

f) optionally sieving of the granulated product

Preferably erythritol is turbomilled to obtain a volume mean diameter of less than 100 μm, preferably less than 50 μm, more preferably less than 40 μm. The thus obtained product has a specific surface area greater than 0.25 m²/g, preferably greater than 0.3 m²/g, more preferably greater than 0.4 m²/g, and it turns out to have an additional positive effect on the subsequent granulation.

The binder, isomalt can be added in dry or liquid form. The specific surface area of the dry isomalt may have an effect on the subsequent granulation. When adding isomalt in dry form, water is further added. Based upon the total dry matter of erythritol and isomalt, water is added in quantities of from 2% to 10%, preferably from 3% to 8%, most preferably in quantities at about 6% to 7%.

Depending upon the volume mean diameter and the moisture content of the blend, the product is sieved and/or dried.

The granules formed in step c) of the current process are optionally pressed through a sieve of a predetermined size. Preferably a screening machine is applied for this sieving. At the same time or thereafter the product is dried.

Any drier type can be applied for drying of the granules, but preferably a fluid bed is applied for this purpose. The sufficiently dry product is granulated in a typical granulator.

It further describes a process for preparing the tablet according to the current invention and it comprises the following steps:

a) Taking the granulated product prepared according to the current invention,

b) Blending with a lubricant,

c) Tabletting at compressing forces from 5 to 20 kN.

The granulated product (=compressible composition) is further blended with a suitable lubricant, preferably magnesium stearate and tabletted in a tabletting machine.

Finally it relates to the use of tablet in food, feed, pharma and cosmetic applications.

If tablets are prepared for food (confectionery) applications than in general up to about 99% (w/w) consists of the erythritol containing compressible composition and aroma, colourant, flavour and a lubricating agent, are added. If tablets are prepared for pharmaceutical applications an active ingredient such as a drug is added and fillers, lubricating agents or disintegrating agents are added if needed.

The invention will hereunder be illustrated in the form of the following examples.

### EXAMPLES

Methods for evaluating granule and tablet properties

The granules were characterized by their volume mean diameter (size distribution).

The following measurement method was employed.

Size distribution. Size distribution was determined according to the European Pharmacopoeia VI Test method 2.9.31 using a laser light particle sizer, type Helos KF—Rodos T4.1, of Sympatec GmbH (Germany). The particle size was analysed by laser light diffraction.

The tablets were characterized by their hardness and friability. For each compression force, 10 tablets for hardness and 19 tablets for friability were analyzed and mean values were calculated. The following measuring methods were employed.

Hardness. Hardness, i.e. the diametral crushing strength, was determined according to the European Pharmacopoeia VI Test method 2.9.8 Resistance to crushing of tablets by using a conventional pharmaceutical hardness tester (hardness tester model Multicheck V, available from Erweka GmbH (Germany)). In order to compare values across different size tablets, the breaking strength was normalized for the area of the break. The normalized value, expressed as N/mm², is herein referred to as tensile strength (Ts) and calculated as follows:

Ts=2H/πTD,

wherein H is the hardness, T the thickness and D the diameter of the tablet. For each compression force, 10 tablets were analyzed on hardness (H), thickness (T) and diameter (D).

Friability. Friability measurements were determined according to the European Pharmacopoeia VI Test method 2.9.7 Friability of uncoated tablets.

### Moisture Absorption

### Example 1

Coarse erythritol product (Cargill Zerose™ 16952) was milled in a Bauermeister turbo mill UTL at a 1 mm sieve and powder with a volume mean diameter of 20 μm was obtained. The volume mean diameter was determined with laser diffraction. The erythritol had a specific surface area of 0.45 m²/g.

400 g of the milled erythritol powder was dry blended in a high Shear Mixer (Pro-C-ept-Mi-Pro, Chopper: 3000 rpm and Impeller: 1200 rpm) with 100 g isomalt (Cargill C*PharmlsoMaltidex™ new grade 2009) for 10 seconds.

34.4 ml of water was added in droplets at 10 ml/min. After the addition of the liquid, the mixing of the blend was continued for 60 seconds.

The granulated powder was manually wet screened over a 2 mm sieve.

The wet sieved granules were dried in the fluid bed (Aeromatic-Fielder GEA—Strea-1) for 30 minutes at a temperature of 70° C.

The dried granules were screened in the granulator (Erweka (FGS+AR400E)) over a sieve of 0.315 mm for 5 to 10 minutes at 100 turns per minute

The dry sieved granules were then blended with 1% of magnesium stearate in a Pharmatech Equipment at 28 rpm.

### Example 2

The granulated product obtained in example 1 was then tabletted in a tabletting machine (Korsch-PH100) at compression forces varying from 5 kN to 20 kN.

Tablets had a surface of 1 cm², the diameter of the tablet was 11.3 mm and the weight is 350 mg.

The thus obtained tablets and granulated product were analyzed as follows:

The granulated product of example 1 had a volume mean diameter of 181 μm.

### Friability

| Compression Force | Product from example 2 |
|---|---|
| (kN) | % |
| 5 | 0.28 |
| 10 | 0.17 |
| 15 | 0.14 |
| 20 | 0.31 |

### Tensile Strength

| Compression Force | Product from example 2 |
|---|---|
| (kN) | (N/mm²) |
| 5 | 1.21 |
| 10 | 2.34 |
| 15 | 3.18 |
| 20 | 4.05 |

### Hardness

| Compression Force | Product from example 2 |
|---|---|
| (kN) | (N) |
| 5 | 66 |
| 10 | 119 |
| 15 | 157 |
| 20 | 196 |

### Example 3

Example 1 and 2 were repeated but instead of using isomalt (Cargill C*PharmlsoMaltidex™ new grade 2009) isomalt (C*IsoMaltidex 16506 from Cargill was applied. The recipe, the procedure as well as the outcome of the experiments was exactly the same as laid out in example 1 and 2.

What is claimed is:

1. A granulated compressible composition consisting of erythritol and from 10% w/w to 50% w/w isomalt.

2. The composition of claim 1, wherein the erythritol has a specific surface area greater than 0.25 m²/g.

3. The composition of claim 1, wherein the composition has a moisture pick-up below 0.5% at 65% relative humidity, at 25° C.

4. A chewable tablet comprising the composition of claim 1.

5. The tablet of claim 4, wherein the tablet has a friability of 0.1 to 0.4%, at a compression force of from 5 to 20 kN.

6. The tablet of claim 4, wherein the tablet has a tensile strength at 20 kN of at least 3.5 N/mm².

7. A process for preparing a compressible composition of claim 1, the process comprising:
a) taking erythritol;
b) adding isomalt in dry or liquid form, optionally adding water;
c) granulating;
d) optionally wet sieving the granulated product;
e) drying the granulated product; and
f) optionally sieving of the granulated product.

8. A process for preparing the tablet of claim 4, the process comprising to:
a) taking the granulated product prepared according to the process of claim 7;
b) blending the granulated product with a lubricant to form a mixture; and
c) tabletting the mixture at compressing forces varying from 5 to 20 kN.

9. The process of claim 8, wherein an active ingredient is added in step a) and/or b).

10. The tablet of claim 4, further comprising at least one of an aroma, a colorant, a flavor, a lubricating agent, an active ingredient, or a disintegrating agent.

11. The tablet of claim 4, wherein the erythritol has a specific surface area greater than 0.25 m²/g.

12. The tablet of claim 4, wherein the composition has a moisture pick-up below 0.5% at 65% relative humidity, at 25° C.
